(19) United States
(12) Patent Application Publication
(10) Pub. No.: US 2005/0148552 A1
(43) Pub. Date: Jul. 7, 2005

(54) Methods of treating eczema

(76) Inventors: Maria Emanuel Ryan, Laurel Hollow, NY (US); Yu Liu, Guangzhou (CN); Lorne M. Golub, Smithtown, NY (US); Hsi-Ming Lee, Setauket, NY (US); Sanford R. Simon, Stony Brook, NY (US); Stephen G. Walker, East Setauket, NY (US)

Correspondence Address: HOFFMANN & BARON, LLP, 6900 JERICHO TURNPIKE, SYOSSET, NY, 11791, US

(21) Appl. No.: 10/982,979

(22) Filed: Nov. 5, 2004

Related U.S. Application Data
(60) Provisional application No. 60/518,354, filed on Nov. 6, 2003.

(51) Int. Cl. A61K 31/65

(52) U.S. Cl. 514/152

(57) ABSTRACT

A method of treating eczema in a human or other mammal in need thereof comprising administering systemically to said human or other mammal a tetracycline compound in an amount that is effective to treat eczema, but has substantially no antibacterial activity.

This application asserts priority to U.S. Provisional Application Ser. No. 60/518,354, filed on Nov. 6, 2003. The specification of U.S. Provisional Application Ser. No. 60/518,354 if hereby incorporated by reference in its entirety.

The present invention was made with Government support under Grant No. 1R21DE14491-01 awarded by the National Institute of Health. The Government has certain rights in the invention.

### BACKGROUND OF THE INVENTION

Eczema, also known as atopic dermatitis, involves inflammation of the skin. The condition is characterized by scaley or crusty patches of skin, often accompanied by redness, blistering, and itching. Significant discomfort in humans and other mammals often accompanies eczema.

Microorganisms, especially Propionibacterium acnes, are strongly implicated in the pathogenesis of acne and acne-associated skin orders. The microorganisms are thought to release microbial mediators of inflammation into the dermis, or to trigger the release of cytokines from ductal keratinocytes.

There are numerous references that disclose various drugs for treating eczema. Eczema is often accompanied by bacterial infections. Therefore, the above drugs often include antibiotic agents as additional ingredients.

For example, tetracycline antibiotics are often used as such additional ingredients in the treatment of eczema in situations where the eczema is accompanied by bacterial infection. See for example, U.S. Pat. No. 5,057,501 to Thomfeldt, for the use of sesquiterpene compounds; U.S. Pat. No. 6,180,662 to Lanzendorfer, et al., for the use of flavonoids; and U.S. Pat. No. 6,486,165 to Zhang, et al., for the use of kappa agonist compounds.

Many other references, such as U.S. Pat. No. 5,720,948 to Brucks, et al., U.S. Pat. No. 5,061,700 to Dow, et al., and U.S. Pat. No. 6,309,669 to Setterstrom, et al., disclose drug delivery vehicles that can be useful in the treatment of various conditions, including eczema. Such delivery vehicles are said to include various drugs. For example, antibiotic agents such as tetracycline antibiotics may be included within the delivery vehicles.

Eczema is often accompanied by dermal disorders associated with acne. Such eczemas in the presence of acne are referred to herein as acne-associated eczemas.

Tetracycline antibiotics are well-known for treating acne and acne-associated disorders. Acne and its associated disorders are characterized by various types of lesions. The areas affected typically are areas of the skin where sebaceous glands are largest, most numerous, and most active.

Accordingly, the efficacy of antibiotics in treating acne and its associated disorders is thought to be due, in significant part, to their direct inhibitory effect on the growth and metabolism of microorganisms. Systemically-administered tetracycline antibiotics, especially minocycline hydrochloride, are particularly effective in treating bacterial infection associated with acne.

The compound tetracycline is a member of a class of antibiotic compounds that is referred to as the tetracyclines, tetracycline compounds, tetracycline derivatives, and the like. The parent compound, tetracycline, has the following structure:

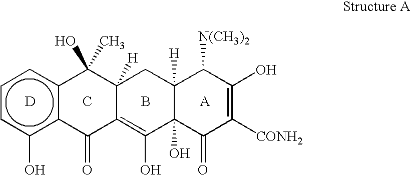

The numbering system of the multiple ring nucleus is as follows:

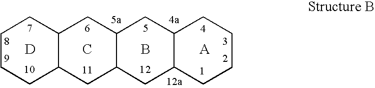

Tetracycline, as well as the terramycin and aureomycin derivatives, exist in nature, and are well known antibiotics. Natural tetracyclines may be modified without losing their antibiotic properties, although certain elements must be retained. The modifications that may and may not be made to the basic tetracycline structure have been reviewed by Mitscher in The Chemistry of Tetracyclines, Chapter 6, Marcel Dekker, Publishers, New York (1978).

According to Mitscher, the substituents at positions 5-9 of the tetracycline ring system may be modified without the complete loss of antibiotic properties. Changes to the basic ring system or replacement of the substituents at positions 4 and 10-12a generally lead to synthetic tetracyclines with substantially less or effectively no antibacterial activity. Non-antibacterial chemically modified tetracyclines are referred to herein as CMTs.

The activity of tetracycline antibiotics is generally proportional to the dose administered. Accordingly, in moderate to severe forms of infections, oral tetracycline antibacterials are typically administered at high doses. For example, in conventional acne or acne-associated eczema therapy, tetracycline is administered at an initial dose of 500 to 2,000 mg/day, followed by a maintenance dose of 250-500 mg/day.

In addition to their direct antibiotic activity, further activities of tetracycline antibacterials have been investigated for possible therapeutic effects on skin disorders. For example, Plewig et al., disclose experiments designed to test the hypothesis that antimicrobial agents are effective in treating inflammatory dermatoses. Journal of Investigative Dermatology 65: 532 (1975). The experiments of Plewig et al. establish that tetracycline antibiotics administered in antibiotic doses have anti-inflammatory properties in treating pustules induced by potassium iodide patches.

Similarly, Elewski et al., speculate that the therapeutic effect of tetracycline antibiotics in skin diseases associated with bacteria, e.g., acne, may be due to inhibition of bacterially induced inflammation in addition to the direct antibacterial effect. J. Amer. Acad. Dermatol., 8: 807-812 (1983).

As can be seen from the discussion above, the prior art discloses the use of tetracycline antibiotics at antibiotic doses in the treatment of inflammatory skin conditions. Although the tetracycline antibiotics are reported to have beneficial secondary effects, the ultimate purpose of using the tetracycline antibiotics has always been to treat the bacterial infection.

The use of antibiotics in the treatment of acne and its associated disorders, however, can lead to undesirable side effects. For example, the long term administration of tetracycline antibiotics can reduce or eliminate healthy microbial flora, such as intestinal and vaginal flora, and can lead to the production of antibiotic-resistant organisms and the overgrowth of yeast and fungi.

In view of the above shortcomings of the use of tetracycline antibiotics, a method of treating acne and acne-associated skin disorders by systemic administration of non-antibacterial tetracycline formulations was previously disclosed in published U.S. application 2003/0139380 assigned to CollaGenex Pharmaceuticals, Inc. of Newtown, Pa. One of the acne-associated skin disorders was said to be acne-associated eczema, i.e., seborrhoic dermatitis in the presence of acne.

None of the above references address the use of non-antibacterial tetracycline formulations for treating eczema, and especially eczema that does not accompany acne, i.e., non-acne-associated eczema. Accordingly, there is a need for an effective treatment of eczema, and especially non-acne-associated eczema, in which the therapeutic effects of the tetracyclines can be used without the undesirable side effects produced by the usual administration of antibacterials for combating bacterial infection.

### SUMMARY OF THE INVENTION

The present invention provides a method of treating eczema in a mammal in need thereof. The method comprises administering to the mammal an effective amount of a non-antibacterial tetracycline formulation, i.e., either a sub-antibacterial dose of an antibiotic tetracycline compound, or a non-antibacterial tetracycline compound. In another embodiment, the eczema is non-acne-associated eczema.

### DETAILED DESCRIPTION

The present invention provides a method of treating eczema. As used herein, the term “eczema” is a disorder of the skin characterized by scaley or crusty patches of skin, often accompanied by redness, blistering, and itching, and perhaps blemishes or skin lesions. These blemishes and lesions are often accompanied by inflammation of the skin glands and pilosebaceous follicles, as well as, microbial, especially bacterial, infection.

For the purposes of this specification, eczema includes all types of eczema. The eczema may or may not accompany acne. In one embodiment, the eczema does not accompany acne, i.e., non-acne-associated eczema.

Some types of eczema that can be treated in accordance with the present invention include, for example, atopic eczema, contact eczema, seborrheic eczema, nummular eczema, neurodermatitis, stasis dermatitis, or dyshidrotic eczema.

Atopic eczema is a hereditary predisposition for inflammation in the skin.

Contact eczema is a general term for an inflamed skin condition caused by contact of the skin to an irritant or allergen. Hence, specific forms of contact eczema include allergic contact eczema and irritant contact eczema.

Seborrheic eczema, also known as seborrhoea, or seborrheic dermatitis, refers to eczema predominantly of the scalp, but may affect other parts of the body. Seborrheic eczema is often associated with dandruff, scaling, and redness. Seborrheic eczema is also known as seborrheic dermatitis.

Nummular eczema, also known as nummular eczematous dermatitis, or discoid eczema, is characterized by coin-shaped lesions on the skin. The cause of the lesions may be dry skin in low humidity environments, or bacterial infections that induce hypersensitivity in the skin.

Neurodermatitis is a chronic type of eczema, characterized by raised, rough, itchy patches of skin, typically on the neck, wrist, and ankles. Possible causes of neurodermatitis include sensitization of the skin over time by an external agent, or by stress, anxiety, dry skin, or infection.

Stasis dermatitis is characterized by a red, itchy rash on the lower legs. Stasis dermatitis may be transformed into a serious condition in which the legs swell. The common cause of stasis dermatitis is poor blood flow from the legs to the heart.

Dyshidrotic eczema, also known as dyshidrosis, or pompholyx, is characterized by the formation of small blisters on the skin that cause intense itching. The blisters may be transformed into an intensely itchy rash. Dyshidrotic eczema normally develops on the hands and feet. A possible cause of dyshidrotic eczema is an inherited allergic response in the skin.

The method of the present invention comprises the administration of a non-antibacterial tetracycline formulation to a mammal with eczema. The class of tetracycline compounds, including tetracycline itself, was described in the background section of this specification.

The non-antibacterial tetracycline formulation is administered to a mammal in an amount that is effective for the treatment of eczema. The treatment is considered effective if there is a reduction or inhibition of the redness, patchiness, itchiness, blemishes and/or lesions associated with eczema. The actual preferred amounts of the non-antibacterial tetracycline formulation in a specified case will vary according to the type and severity of the eczema being treated, the particular composition formulated, the mode of application, and the particular subject being treated. The appropriate amount of the non-antibacterial tetracycline formulation can readily be determined by those skilled in the art.

The non-antibacterial tetracycline formulation is either a sub-antibacterial dose of an antibiotic tetracycline compound, or a non-antibacterial tetracycline compound. Antibiotic tetracycline compounds are administered in an effective amount that has no antibacterial activity, i.e., administered below the minimum antibacterial serum concentration. Such an amount is referred to herein as a “sub-antibacterial dose” or a “sub-antibacterial amount.”

The dose of the non-antibacterial tetracycline formulation may be based on a per day basis, i.e., mg/day. Alternatively, the dose of the non-antibacterial tetracycline formulation may be based on serum level concentration. For purposes of this application, “serum level” means the concentration of the non-antibacterial tetracycline formulation in a patient's blood twenty four hours after the dose taken on day seven of a treatment regimen.

Some examples of antibiotic tetracycline compounds include doxycycline, minocycline, tetracycline, oxytetracycline, chlortetracycline, demeclocycline, lymecycline and their pharmaceutically acceptable salts. For example, doxycycline is preferably administered as its hyclate salt or as a hydrate, preferably a monohydrate.

Some examples of antibiotic amounts of members of the tetracycline family include 100 mg/day of doxycycline, 100 mg/day of minocycline, 250 mg of tetracycline four times a day, 1000 mg/day of oxytetracycline, 600 mg/day of demeclocycline and 600 mg/day of lymecycline.

Sub-antibacterial amounts of antibiotic tetracycline compounds may be administered in a minimum amount which is approximately 10%, preferably about 25%, and more preferably about 40% of the minimum antibacterial amount. The maximum sub-antibacterial amounts of antibacterial tetracycline compounds is approximately about 80%, preferably about 70%, and more preferably about 60% of the antibacterial amount.

Some examples of suitable sub-antibacterial doses of antibacterial tetracyclines based on steady-state pharmacokinetics include: 20 mg/twice a day for doxycycline; 38 mg of minocycline one, two, three or four times a day; and 60 mg of tetracycline one, two, three or four times a day.

When the amount of an antibiotic tetracycline compound administered is based on serum level, the tetracycline compound is preferably administered in an amount that results in a minimum serum tetracycline concentration of about 10%, preferably about 25%, and more preferably about 40% of the antibacterial amount. The tetracycline compound is also preferably administered in an amount that results in a maximum serum tetracycline concentration of approximately about 80%, preferably about 70%, and more preferably about 60% of the antibacterial amount.

For example, a single dose of two 100 mg minocycline HCl tablets or capsules administered to adult humans results in minocycline serum levels ranging from 0.74 to 4.45 μg/ml over a period of an hour. The average level is 2.24 μg/ml.

Two hundred and fifty milligrams of tetracycline HCl administered every six hours over a twenty-four hour period produces a peak serum level of approximately 3 μg/ml. Five hundred milligrams of tetracycline HCl administered every six hours over a twenty-four hour period produces a serum concentration level of 4 to 5 μg/ml.

As stated earlier, the non-antibacterial tetracycline formulation also can comprise a non-antibacterial tetracycline compound. Non-antibacterial tetracycline compounds are structurally related to the antibiotic tetracyclines, but have had their antibacterial activity substantially or completely eliminated by chemical modification. The term “substantially” as used herein means that even though a small number of more sensitive bacterial cells may be inhibited, the inhibition is not clinically significant.

Tetracycline compounds are considered to be non-antibiotic when they are capable of achieving antibacterial activity comparable to that of doxycycline only at concentrations at least about ten times, preferably at least about twenty five times, greater than that of doxycycline.

Examples of chemically modified non-antibacterial tetracyclines (CMTs) include those that lack the dimethylamino group at position 4 of the tetracycline ring structure, e.g.,:
4-dedimethylaminotetracycline (CMT-1),
6-demethyl-6-deoxy-4-de(dimethylamino)tetracycline (CMT-3),
7-chloro-4-de(dimethylamino)tetracycline (CMT-4),
4-hydroxy-4-de(dimethylamino)tetracycline (CMT-6),
4-de(dimethylamino)-12α-deoxytetracycline (CMT-7),
6-deoxy-5α-hydroxy-4-de(dimethylamino)tetracycline (CMT-8),
4-dedimethylamino-12α-deoxyanhydrotetracycline (CMT-9),
7-dimethylamino-6-demethyl-6-deoxy-4-de(dimethylamino)tetracycline (CMT-10),
4-dedimethylamino-5-oxytetracycline,
5α,6-anhydro-4-hydroxy-4-de(dimethylamino)tetracycline,
4-de(dimethylamino)-11-hydroxy-12α-deoxytetracycline,
12α-deoxy-4-deoxy-4-de(dimethylamino)tetracycline, and
12α;4α-anhydro-4-de(dimethylamino)tetracycline.

Further examples of tetracyclines modified for reduced antibacterial activity include 6-α-benzylthiomethylenetetracycline, the mono-N-alkylated amide of tetracycline, 6-fluoro-6-demethyltetracycline, 11α-chlorotetracycline, tetracyclinonitrile (CMT-2), and tetracycline pyrazole (CMT-5).

Derivatives of the CMTs mentioned above can also be used. Such derivatives may have a substituent other than hydrogen at the 7, 8, or 9 position of ring D of the tetracycline ring nucleus. Some examples of substituents include halo (e.g., F, Cl, Br, and I); nitro; hydroxy, alkyl carbonyl; alkyl carbonyloxy; alkyl amido; amino; alkyl amino; dialkyl amino; phenyl, carboxylate, etc., wherein alkyl represents C₁-C₁₆, preferably C₁-C₄, straight chain or branched alkyl (e.g., methyl, ethyl, isopropyl).

For example, some derivatives of CMT-3 include:

| | |
|---|---|
| CMT-301 | 7-bromo-6-demethyl-6-deoxy-4-dedimethylaminotetracycline |
| CMT-302 | 7-nitro-6-demethyl-6-deoxy-4-dedimethylaminotetracycline |
| CMT-303 | 9-nitro-6-demethyl-6-deoxy-4-dedimethylaminotetracycline |
| CMT-304 | 7-acetamido-6-demethyl-6-deoxy-4- |
| | dedimethylaminotetracycline |
| CMT-305 | 9-acetamido-6-demethyl-6-deoxy-4- |
| | dedimethylaminotetracycline |
| CMT-306 | 9-dimethylamino-6-demethyl-6-deoxy-4- |
| | dedimethylaminotetracycline |
| CMT-307 | 7-amino-6-demethyl-6-deoxy-4- |
| | dedimethylaminotetracycline |
| CMT-308 | 9-amino-6-demethyl-6-deoxy-4- |
| | dedimethylaminotetracycline |
| CMT-309 | 9-dimethylaminoacetamido-6-demethyl-6-deoxy-4- |
| | dedimethylaminotetracycline |
| CMT-310 | 7-dimethylamino-6-demethyl-6-deoxy-4- |
| | dedimethylaminotetracycline |
| CMT-311 | 9-palmitamide-6-demethyl-6-deoxy-4- |
| | dedimethylaminotetracycline |
| CMT-312 | 2-CONHCH₂-pyrrolidin-1-yl-6-demethyl-6-deoxy-4- |
| | dedimethylaminotetracycline |
| CMT-313 | 2-CONHCH₂-piperidin-1-yl-6-demethyl-6-deoxy-4- |
| | dedimethylaminotetracycline |
| CMT-314 | 2-CONHCH₂-morpholin-1-yl-6-demethyl-6-deoxy-4- |
| | dedimethylaminotetracycline |
| CMT-315 | 2-CONHCH₂-piperazin-1-yl-6-demethyl-6-deoxy-4- |
| | dedimethylaminotetracycline |

Some derivatives of CMT-8 include:

| | |
|---|---|
| CMT-801 | 9-acetamido-4-dedimethylaminodoxycycline |
| CMT-802 | 9-dimethylaminoacetamido-4-dedimethylaminodoxycycline |
| CMT-803 | 9-palmitamide-4-dedimethylaminodoxycycline |
| CMT-804 | 9-nitro-4-dedimethylaminodoxycycline |
| CMT-805 | 9-amino-4-dedimethylaminodoxycycline |
| CMT-806 | 9-dimethylamino-4-dedimethylaminodoxycycline |
| CMT-807 | 2-CONHCH₂-pyrrolidin-1-yl-4-dedimethylaminodoxycycline |
| CMT-808 | 2-CONHCH₂-piperidin-1-yl-4-dedimethylaminodoxycycline |
| CMT-809 | 2-CONHCH₂-piperazin-1-yl-4-dedimethylaminodoxycycline |

Some derivatives of CMT-10 include:

| | |
|---|---|
| CMT-1001 | 7-trimethylammonium-4-dedimethylaminosancycline |
| CMT-1002 | 9-nitro-4-dedimethylaminominocycline |

Further examples of generic and specific tetracycline compounds that are suitable for use in the methods of the invention are disclosed in international PCT Application No. WO 01/87823. All such generic and specific compounds disclosed in PCT Application No. WO 01/87823 are hereby incorporated by reference.

The chemically modified tetracycline compounds can be synthesized by any of the methods known in the art. Suitable methods for synthesizing CMTs include, for example, those described in Mitscher (Ibid.), and U.S. Pat. Nos. 4,704,383, 5,532,227, and 6,506,740.

The minimum amount of a non-antibacterial tetracycline compound administered to a mammal is the lowest amount effective for treating eczema. A suitable minimum amount of a non-antibacterial tetracycline compound is an amount that results in a serum level of about 0.1 μg/ml, and more preferably about 0.5 μg/ml. A suitable minimum daily dose of a non-antibacterial tetracycline compound is about 1 mg/day, more preferably about 20 mg/day, more preferably about 30 mg/day, and even more preferably about 40 mg/day.

The maximum amount of a non-antibacterial tetracycline compound administered to a mammal is the highest effective amount that does not cause undesirable side effects. A suitable maximum amount of a non-antibacterial tetracycline compound is an amount that results in a serum level of about 10 μg/ml, more preferably about 8 μg/ml, more preferably about 6 μg/ml, more preferably about 4 μg/ml, and even more preferably about 1 μg/ml. A suitable maximum daily dose of a non-antibacterial tetracycline compound is about 200 mg/day, more preferably about 100 mg/day, more preferably about 80 mg/day, and even more preferably about 60 mg/day.

Any minimum dosage amount based on serum level described above can be combined with any maximum dosage amount based on serum level described above to form a suitable dosage range based on serum level. Likewise, any minimum daily dosage amount described above can be combined with any maximum daily dosage amount described above to form a suitable daily dosage range.

For example, in one embodiment, 6-demethyl-6-deoxy-4-de(dimethylamino)tetracycline (CMT-3) is administered in doses of about 40 mg/day to about 200 mg/day, or in amounts that result in serum levels of about 1.55 μg/ml to about 10 μg/ml. In another embodiment, CMT-3 is administered in doses of, for example, 1 mg/day to about 12 mg/day, or in amounts that result in serum levels of about 0.1 μg/ml to about 1.1 μg/ml.

The tetracycline formulation may be administered alone or as an adjunct with additional drugs. The additional drugs may or may not be related to the treatment of eczemaper se. Examples of such additional drugs include analgesics, such as aspirin, acetaminophen, ibuprofen, and codeine; corticosteroids such as cortisone, methylprednisolone, prednisone, prednisolone, and dexamethasone; muscle relaxants such as methocarbamol, orphenanidrine, carisoprodol, meprobamate, and diazepam; analeptics such as caffeine, methylphenidate and pentylenetetrazol; antihistamines such as chlorpheniramine, cyproheptadine, promethazine and pyrilamine; and anaesthetic agents such as a morphine derivative, lidocaine, procaine, bupivacaine, or prilocalne. Some other classes of additional drugs include, for example, antibiotics, retinoids, antivirals, and antifungals.

The tetracycline formulations may be administered by any method known in the art. Some examples of suitable modes of administration include oral, systemic, and topical administration.

The tetracyclines can be administered orally by any method known in the art. Liquid or solid oral formulations may be used. Some examples of formulations suitable for oral administration include tablets, capsules, pills, troches, elixirs, suspensions, and syrups.

Systemic administration includes enteral or parenteral modes of administration, e.g., intravenous; intramuscular; subcutaneous, as injectable solutions or suspensions; or intraperitoneal.

For example, the administration can be intranasal, in the form of, for example, a nebulizer, liquid mist, or intranasal spray. The administration can also be transdermal, in the form of, for example, a patch. Alternatively, the administration can be rectal, in the form of, for example, a suppository. Furthermore, the administration can be intrabronchial, in the form of, for example, an inhaler spray.

The administration can also be topical. Topical application of non-antibacterial tetracycline compounds are effective in treating eczema, while not inducing significant toxicity in mammals. For example, amounts of up to about 25% (w/w) in a vehicle are effective. Amounts of from about 0.1% to about 10% are preferred.

Particular non-antibacterial tetracycline compounds have only limited biodistribution, e.g. CMT-5. In such cases, topical application is the preferred method of administration of the compound.

The tetracycline compound may be administered once a day, or more than once a day. For example, the tetracycline compound may be administered 1-6 times a day, preferably 1-2 times a day.

Alternatively, the tetracycline compound may be administered by controlled release. Controlled release administration is a method of drug delivery to achieve a certain level of the drug over a particular period of time. The level typically is measured by serum concentration. For example, 40 milligrams of doxycycline may be administered by controlled release over a 24 hour period.

Further description of methods for delivering tetracycline compounds by controlled release can be found in PCT Application No. WO 02/083106. The aforementioned application is incorporated herein by reference in its entirety.

Combined or coordinated topical and systemic administration of the tetracycline compounds is also contemplated under the invention. For example, a non-absorbable non-antibacterial tetracycline compound can be administered topically, while a tetracycline compound capable of substantial absorption and effective systemic distribution can be administered systemically.

The tetracycline compounds can be in the form of pharmaceutically acceptable salts of the compounds. The term “pharmaceutically acceptable salt” refers to a salt prepared from a suitable tetracycline compound and, for example, an acid. The salt is acceptably non-toxic and has acceptable pharmacokinetics. Such salts are formed by well known procedures.

Suitable acids for producing salts of the tetracycline compounds include mineral acids and organic acids. Some examples of mineral acids include hydrochloric, hydriodic, hydrobromic, phosphoric, metaphosphoric, nitric and sulfuric acids. Some examples of organic acids include tartaric, acetic, citric, malic, benzoic, glycollic, gluconic, gulonic, succinic, arylsulfonic, e.g., p-toluenesulfonic acids, and the like.

For the pharmaceutical purposes described above, the tetracycline compounds of the invention can be formulated in pharmaceutical preparations optionally with a suitable pharmaceutical carrier (vehicle) or excipient as understood by practitioners in the art. In this specification, a pharmaceutical carrier is considered to be synonymous with a vehicle or an excipient as is understood by practitioners in the art. Examples of carriers include starch, milk, sugar, certain types of clay, gelatin, stearic acid or salts thereof, magnesium or calcium stearate, talc, vegetable fats or oils, gums and glycols.

The tetracycline formulations may also comprise one or more of the following: a stabilizer, a surfactant, preferably a nonionic surfactant, and optionally a salt and/or a buffering agent.

The stabilizer may, for example, be an amino acid, such as for instance, glycine; or an oligosaccharide, such as for example, sucrose, tetralose, lactose or a dextran. Alternatively, the stabilizer may be a sugar alcohol, such as for instance, mannitol; or a combination thereof. Preferably the stabilizer or combination of stabilizers constitutes from about 0.1% to about 10% weight for weight of the tetracycline compound.

The surfactant is preferably a nonionic surfactant, such as a polysorbate. Some examples of suitable surfactants include Tween 20, Tween 80; a polyethylene glycol or a polyoxyethylene polyoxypropylene glycol, such as Pluronic F-68 at from about 0.001% (w/v) to about 10% (w/v).

The salt or buffering agent may be any salt or buffering agent, such as, for example, sodium chloride, or sodium/potassium phosphate, respectively. Preferably, the buffering agent maintains the pH of the tetracycline formulation in the range of about 5.5 to about 7.5. The salt and/or buffering agent is also useful to maintain the osmolality at a level suitable for administration to a mammal. Preferably the salt or buffering agent is present at a roughly isotonic concentration of about 150 mM to about 300 mM.

The tetracycline formulations may additionally contain one or more conventional additives. Some examples of such additives include a solubilizer such as, for example, glycerol; an antioxidant such as, for example, benzalkonium chloride (a mixture of quaternary ammonium compounds, known as “quart”), benzyl alcohol, chloretone or chlorobutanol; or an isotonic agent or buffering agent, such as described above. As a further precaution against oxidation or other spoilage, the tetracycline formulations may be stored under nitrogen gas in vials sealed with impermeable stoppers.

When aqueous suspensions are used for oral administration, emulsifying and/or suspending agents are commonly added. In addition, coloring, sweetening and/or flavoring agents may be added to the oral compositions.

For various modes of administration, e.g., intramuscular, intraperitoneal, subcutaneous and intravenous, sterile solutions of the tetracycline compounds are preferably employed, and the pH of the solutions suitably adjusted and buffered. For intravenous use, the total concentration of the solute(s) can be controlled in order to render the preparation isotonic.

Carrier compositions deemed to be suited for topical use include gels, salves, lotions, creams, ointments and the like. The non-antibacterial tetracycline compound can also be incorporated with a support base or matrix or the like which can be directly applied to skin.

Any mammal capable of suffering from eczema can be treated in accordance with the present invention. Mammals include, for example, humans, baboons, and other primates, as well as pet animals such as dogs and cats, laboratory animals such as rats and mice, and farm animals such as horses, sheep, and cows.

Thus, whereas there have been described what are presently believed to be the preferred embodiments of the present invention, those skilled in the art will realize that other and further embodiments can be made without departing from the spirit of the invention, and it is intended to include all such further modifications and changes as come within the true scope of the claims set forth herein.

1. A method for treating eczema in a mammal in need thereof, the method comprising administering to the mammal an effective amount of a non-antibacterial tetracycline formulation.

2. A method according to claim 1, wherein the mammal is a human.

3. A method according to claim 1, wherein the non-antibacterial tetracycline formulation comprises an antibacterial tetracycline at a sub-antibacterial dose.

4. A method according to claim 1, wherein the non-antibacterial tetracycline formulation comprises a non-antibacterial tetracycline.

5. A method according to claim 3, wherein the antibacterial tetracycline is doxycycline.

6. A method according to claim 3, wherein the antibacterial tetracycline is minocycline.

7. A method according to claim 4, wherein the non-antibacterial tetracycline is CMT-3.

8. A method according to claim 4, wherein the non-antibacterial tetracycline is CMT-308.

9. A method according to claim 4, wherein the non-antibacterial tetracycline is CMT-8.

10. A method according to claim 4, wherein the non-antibacterial tetracycline is CMT-10.

11. A method according to claim 4, wherein the non-antibacterial tetracycline is CMT-801.

12. A method according to claim 4, wherein the non-antibacterial tetracycline is CMT-802.

13. A method according to claim 4, wherein the non-antibacterial tetracycline is CMT-803.

14. A method according to claim 4, wherein the non-antibacterial tetracycline is CMT-804.

15. A method according to claim 4, wherein the non-antibacterial tetracycline is CMT-1002.

16. A method according to claim 1, wherein the eczema is atopic eczema.

17. A method according to claim 1, wherein the eczema is contact eczema.

18. A method according to claim 1, wherein the eczema is allergic contact eczema.

19. A method according to claim 1, wherein the eczema is seborrheic eczema.

20. A method according to claim 1, wherein the eczema is nummular eczema.

21. A method according to claim 1, wherein the eczema is neurodermatitis.

22. A method according to claim 1, wherein the eczema is stasis dermatitis.

23. A method according to claim 1, wherein the eczema is dyshidrotic eczema.

24. A method for treating non-acne-associated eczema in a mammal in need thereof, the method comprising administering to the mammal an effective amount of a non-antibacterial tetracycline formulation.

25. A method according to claim 24, wherein the mammal is a human.

26. A method according to claim 24, wherein the non-antibacterial tetracycline formulation comprises an antibacterial tetracycline at a sub-antibacterial dose.

27. A method according to claim 24, wherein the non-antibacterial tetracycline formulation comprises a non-antibacterial tetracycline.

28. A method according to claim 26, wherein the antibacterial tetracycline is doxycycline.

29. A method according to claim 26, wherein the antibacterial tetracycline is minocycline.

30. A method according to claim 27, wherein the non-antibacterial tetracycline is CMT-3.

31. A method according to claim 27, wherein the non-antibacterial tetracycline is CMT-308.

32. A method according to claim 27, wherein the non-antibacterial tetracycline is CMT-8.

33. A method according to claim 27, wherein the non-antibacterial tetracycline is CMT-10.

34. A method according to claim 27, wherein the non-antibacterial tetracycline is CMT-801.

35. A method according to claim 27, wherein the non-antibacterial tetracycline is CMT-802.

36. A method according to claim 27, wherein the non-antibacterial tetracycline is CMT-803.

37. A method according to claim 27, wherein the non-antibacterial tetracycline is CMT-804.

38. A method according to claim 27, wherein the non-antibacterial tetracycline is CMT-1002.

39. A method according to claim 24, wherein the non-acne-associated eczema is atopic eczema.

40. A method according to claim 24, wherein the non-acne-associated eczema is contact eczema.

41. A method according to claim 24, wherein the non-acne-associated eczema is allergic contact eczema.

42. A method according to claim 24, wherein the non-acne-associated eczema is seborrheic eczema.

43. A method according to claim 24, wherein the non-acne-associated eczema is nummular eczema.

44. A method according to claim 24, wherein the non-acne-associated eczema is neurodermatitis.

45. A method according to claim 24, wherein the non-acne-associated eczema is stasis dermatitis.

46. A method according to claim 24, wherein the non-acne-associated eczema is dyshidrotic eczema.
